(19) United States
(12) Patent Application Publication
(10) Pub. No.: US 2023/0011342 A1
(43) Pub. Date: Jan. 12, 2023

(54) MEDICAL PROCEDURE SCHEDULING SYSTEM

(71) Applicant: Ashvin Dewan, Sugar Land, TX (US)

(72) Inventor: Ashvin Dewan, Sugar Land, TX (US)

(21) Appl. No.: 17/372,989

(22) Filed: Jul. 12, 2021

(51) Int. Cl. G16H 40/20; G06Q 40/08; G06N 5/04; G16H 10/60

(52) U.S. Cl. CPC G16H 40/20; G06Q 40/08; G06N 5/04; G16H 10/60

(57) ABSTRACT

A surgical scheduling system is disclosed comprising a server having a server engine operable to communicate with a surgical scheduling management system having a point of care tool to permit a practitioner to construct at least one case, which includes case data. A repository receives the case data and retrieves requirements for the surgical case. A dashboard disseminates the requirements and transmits the requirements to at least one of a plurality of users via a communication system. A scheduler is in operable communication with a scheduling system to generate a schedule for the case.

### CROSS-REFERENCE TO RELATED APPLICATIONS

The present application claims priority to U.S. Provisional Patent Application 62/987,394 filed on Mar. 10, 2020, entitled “SURGERY SCHEDULING SYSTEM” the entire disclosure of which is incorporated by reference herein.

### TECHNICAL FIELD

The embodiments generally relate to computerized scheduling systems, and more particularly relate to scheduling systems which may be utilized by healthcare professionals and facilities to manage patient scheduling and procedures.

### BACKGROUND

Medical centers are subject to severe financial, organizational and social pressures resulting from the increased demands on services and limitation in healthcare resources. Surgical services offer a complex and resource-intensive operation which results in significant inefficiency. These inefficiencies have long been accepted by medical center managers. Recent advances in technologies for resource management, personnel management and communication systems have provided significant positive impact on the utilization of resources involved in the care of surgical patients.

Surgery is a stressful time for patients, which begins at the initial diagnosis of the problem requiring a procedure. Often surgeons only have at most 10 minutes to explain the processes for the proposed surgery, expectations, outcomes, recovery and potential risks and complications. This results in an overload of information during that short period of time for the patient, who often is unable to process and retain all of the details immediately and subsequently is left with more questions than answers.

For surgeons and the medical staff, the process of surgery is complicated by an overworked staff, time constraints, the interruption of clinic workflow, paper-based case-requests, scheduling, and communication with the various parties of the surgery. Schedulers must manage a high volume of cases while keeping patients and practitioners alike up-to-date.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

A surgical scheduling system is disclosed comprising a server having a server engine operable to communicate with a surgical scheduling management system having a point of care tool to permit a practitioner to construct at least one case, which includes case data. A repository receives the case data and retrieves requirements for the surgical case. A dashboard disseminates the requirements and transmits the requirements to at least one of a plurality of users via a communication system. A scheduler is in operable communication with a scheduling system to generate a schedule for the procedure.

The system allows for the medical professional to input a diagnosis for a patient and select a procedure in view the diagnosis. The medical professional may also use the system to order post-operative equipment (e.g., a brace) which the patient will need during the recovery period. The system allows for efficient and convenient scheduling which may permit the scheduler to operate independently of the physician.

In one aspect, an artificial intelligence engine receives a patient diagnosis and/or medical professional to facilitate the selection of a procedure, post-operative equipment, and/or scheduling of the procedure.

In one aspect, the communication system can be a direct communication between a patient and a practitioner (e.g., the surgeon, members of the surgical team, or other medical staff and personnel). The communication system may allow for the collection of data related to common patient questions and practitioner responses.

In one aspect, the system further comprises a risk analyzer configured to receive the case data, generate a risk profile, and correspond the risk profile to the case.

In one aspect, the system further comprises a notification generator to disseminate notifications to at least one of the plurality of users.

In one aspect, a pre-operation module processes pre-operation information and disseminates the pre-operation information to at least one of the plurality of users.

In one aspect, the system further comprises an operation module to process operation information and disseminate the operation information to at least one of the plurality of users.

In one aspect, the system further comprises a post-operation module to process post-operation information and disseminate the post-operation information to at least one of the plurality of users.

In one aspect, the system further comprises a recovery module to process recovery information and disseminate the recovery information to at least one of the plurality of users.

In one aspect, the system utilizes an external scheduling system to generate a schedule comprising scheduling data for at least one medical facility.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of the network infrastructure, according to some embodiments;

FIG. 2 illustrates a block diagram of the surgical scheduling management system, according to some embodiments;

FIG. 3 illustrates a block diagram of the server engine and modules, according to some embodiments;

FIG. 4 illustrates a screenshot of the diagnosis selection interface, according to some embodiments;

FIG. 5 illustrates a screenshot of the procedure selection interface, according to some embodiments;

FIG. 6 illustrates a screenshot of the case listing interface, wherein the healthcare professional can view their scheduled procedures, according to some embodiments;

FIG. 7 illustrates a screenshot of the calendar interface illustrating a daily calendar including scheduled procedures for each available area within the healthcare facility, according to some embodiments;

FIG. 8 illustrates a screenshot of the reports interface to provide metrics for one or more healthcare facilities, according to some embodiments;

FIG. 9 illustrates a flowchart for the patient scheduling process, according to some embodiments; and

FIG. 10 illustrates a block diagram of the application system, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments described herein provide a system for managing surgical scheduling to provide an efficient means for scheduling a surgery. The system allows personnel of a medical facility to independently manage case scheduling.

The system allows for the reduction of billing, coding, and transcription errors while also decreasing the burden of surgeons and medical staff of a medical facility. Meanwhile the system enhances patient, surgeon, and medical staff satisfaction in the workplace, increases scheduling transparency, efficiency, and communication. The system allows for the scheduler to work independently of the surgeon or other medical professional in order to increase the efficiency of the procedure and scheduling process thereof.

As used herein, the term “healthcare professional” may relate to physicians, administrative assistants (such as those involved in ordering materials and/or the scheduling of healthcare services) nurses, surgical technicians, and others working in the healthcare field which are involved in the scheduling of surgical procedures, consultations, and recovery processes.

As used herein, the term “healthcare service” may include any service or process related to a patient's treatment such as consultations, doctor visits, surgeries, therapy, recovery processes and appointments, etc. The system described herein allows for the efficient and semi-automated scheduling of healthcare services, material and equipment ordering, and the like.

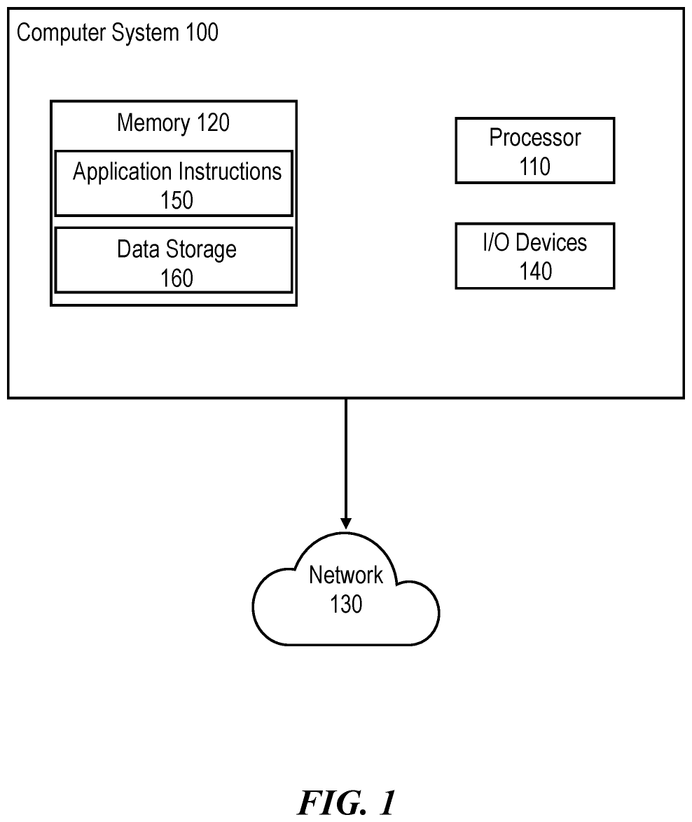

FIG. 1 illustrates a computer system 100, which may be utilized to execute the processes described herein. The computing system 100 is comprised of a standalone computer or mobile computing device, a mainframe computer system, a workstation, a network computer, a desktop computer, a laptop, or the like. The computer system 100 includes one or more processors 110 coupled to a memory 120 via an input/output (I/O) interface. Computer system 100 may further include a network interface to communicate with the network 130. One or more input/output (I/O) devices 140, such as video device(s) (e.g., a camera), audio device(s), and display(s) are in operable communication with the computer system 100. In some embodiments, similar I/O devices 140 may be separate from computer system 100 and may interact with one or more nodes of the computer system 100 through a wired or wireless connection, such as over a network interface.

Processors 110 suitable for the execution of a computer program include both general and special purpose microprocessors and any one or more processors of any digital computing device. The processor 110 will receive instructions and data from a read-only memory or a random-access memory or both. The essential elements of a computing device are a processor for performing actions in accordance with instructions and one or more memory devices for storing instructions and data. Generally, a computing device will also include, or be operatively coupled to receive data from or transfer data to, or both, one or more mass storage devices for storing data, e.g., magnetic, magneto-optical disks, or optical disks; however, a computing device need not have such devices. Moreover, a computing device can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

A network interface may be configured to allow data to be exchanged between the computer system 100 and other devices attached to a network 130, such as other computer systems, or between nodes of the computer system 100. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol.

The memory 120 may include application instructions 150, configured to implement certain embodiments described herein, and a database 160, comprising various data accessible by the application instructions 150. In one embodiment, the application instructions 150 may include software elements corresponding to one or more of the various embodiments described herein. For example, application instructions 150 may be implemented in various embodiments using any desired programming language, scripting language, or combination of programming languages and/or scripting languages (e.g., C, C++, C#, JAVA®, JAVASCRIPT®, PERL®, etc.).

The steps and actions of the computer system 100 described in connection with the embodiments disclosed herein may be embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

Also, any connection may be associated with a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

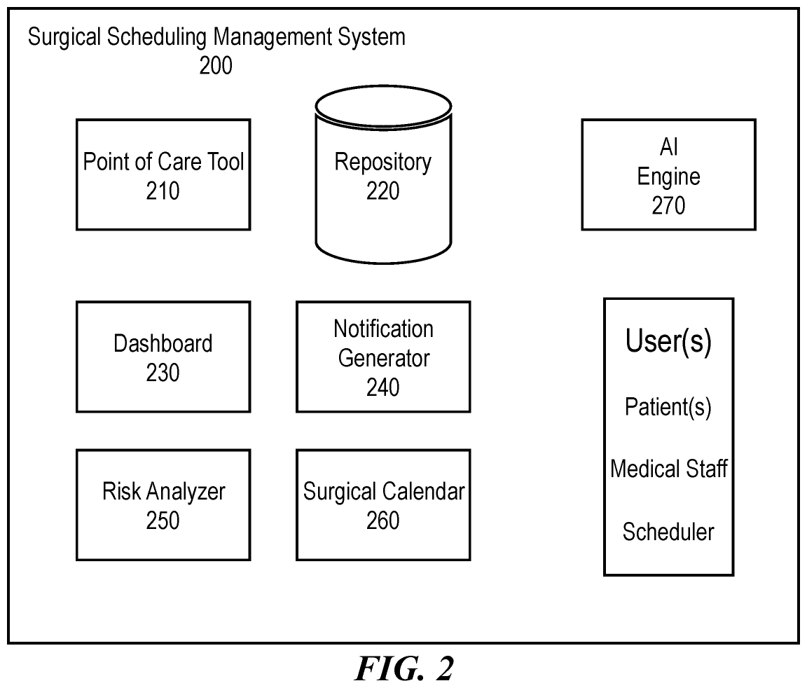

FIG. 2 illustrates a block diagram of the surgical scheduling management system 200, comprising a point of care tool 210 which permits a surgeon to construct a case for each patient; an external system may be utilized to facilitate this process. A repository 220 receives the surgeon's requests, which have been input in the point of care tool, and retrieves the appropriate requirements for the surgical case, which permits an integrated dashboard 230 to receive the requirements of the surgical case and disseminate the information to appropriate parties (e.g., schedulers, case managers, surgeons, etc.). A notification generator 240 transmits communications to various parties such as the patient or the surgeon to update the various parties of information related to the case. A risk analyzer 250 is in communication with the scheduler to allow the scheduler to develop a risk profile of the patient to aid in the identification of opportunities for the medical center and staff to mitigate risks, complications, and readmissions. The scheduler utilizes a surgical calendar 260 to set a schedule for various processes related to the surgery event. An artificial intelligence (AI) engine 270 may receive information from the users of the system (e.g., the medical professional) and process the information to generate an output. For example, the AI engine 270 may interpret a surgeons common procedures and suggest a procedure based on the patient's symptoms in view of the surgeons common procedures. In another example, the AI engine may receive the patient's diagnosis and suggest a procedure based on the diagnosis.

In some embodiments, the AI engine 270 may be used to efficiently schedule procedures based on medical professional availability, patient availability, priority of the patient, operating room availability, etc.

In some embodiments, the AI engine 270 uses aggregated data including historic scheduling data and pooled scheduling data of other similar surgeons scheduling in a similar context to anticipate the surgeon's complex plan.

In some embodiments, the scheduling system, including the AI engine 270, may generate and transmit scheduling and operational recommendations (e.g., by recommending alternative implants). Further, the scheduling system, including the AI engine 270, may provide feedback based on peer scheduling data such as industry best practices or post-operative regimens.

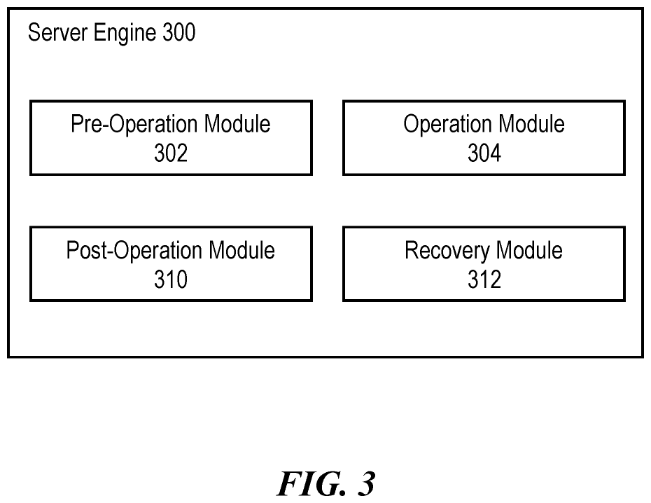

FIG. 3 illustrates a server engine 300 and associated modules including a pre-operation module 302, an operation module 304, a post-operation module 310, and a recovery module 312. Each of the pre-operation module 302, operation module 304, post-operation module 310, and recovery module 312 can be provided as a comprehensive system that can track a patient throughout medical treatment, including through the diagnosis, surgical treatment, and recovery of the patient. Each module may be in communication with various users of the system such as patients, surgeons, medical staff, and other users. Users of the system can include patients and medical practitioners involved with treating one or more of the patients. In some embodiments, the system can be accessible by users other than patients and medical practitioners, such as by medical administrators, e.g., billing administrators, inventory controllers, etc. Different users can have access to different portions of the system, as mentioned above regarding security features. For example, the system may be configured to allow patients to access only the pre-operation 302 and the recovery module 312, to allow medical administrators to access only the operation module 304, and to allow surgeons to access all of the modules. Further, a user may have access to only a portion of a module, e.g., to only a subset of component modules within any one or more of the modules.

It will be appreciated that the system can include security features such that the aspects of the system available to any particular user can be determined based on the identity of the user and/or the location from which the user is accessing the system. To that end, each user can have a unique username, password, and/or other security credentials to facilitate access to the system 200. The received security parameter information can be checked against a database of authorized users to determine whether the user is authorized and to what extent the user is permitted to interact with the system, view information stored in the system, and so forth. Exemplary examples of parties who may be permitted to access the system include patients, potential patients, significant others, friends, and family members of patients or potential patients, surgical technicians, imaging technicians (e.g., x-ray technicians, MRI technicians, etc.), surgeons, nurses, hospital administrators, surgical equipment manufacturer employees, insurance providers, and operating room directors.

In some embodiments, the communication system may be provided as a messaging system between a practitioner (such as the surgeon) and the patient. Further, messages may be aggregated and transmitted to an AI (artificial intelligence) and machine learning engine to determine common questions and responses to the common questions. In such, the communications system may operate to send responses to patients autonomously or semi-autonomously.

In some embodiments, the system provides input to the patient, practitioner, or other user to inform the user of means for saving resources (e.g., a method for saving time of recovery, a device that is more cost effective, etc.) and provide a safer experience for the patient (e.g., suggesting a procedure to the practitioner to increase safety or efficacy).

In some embodiments, the system may operate to regulate the insurance billing and filing process to reduce the likelihood of the denial of insurance. The insurance system may operate independently of the scheduler to further increase the efficiency of the scheduling and insurance approval process.

In some embodiments, the system may automatically seek insurance approval using information in the system. The system may search and retrieve additional relevant information from medical records to automatically provide appropriate information to the insurance provider.

In a further embodiment, an AI and machine learning engine is configured to receive aggregated data regarding the denial or acceptance of insurance to detect patterns in payor denials. In such, the system may alert the user when conditions of a recognized pattern are present such that insurance may likely be denied.

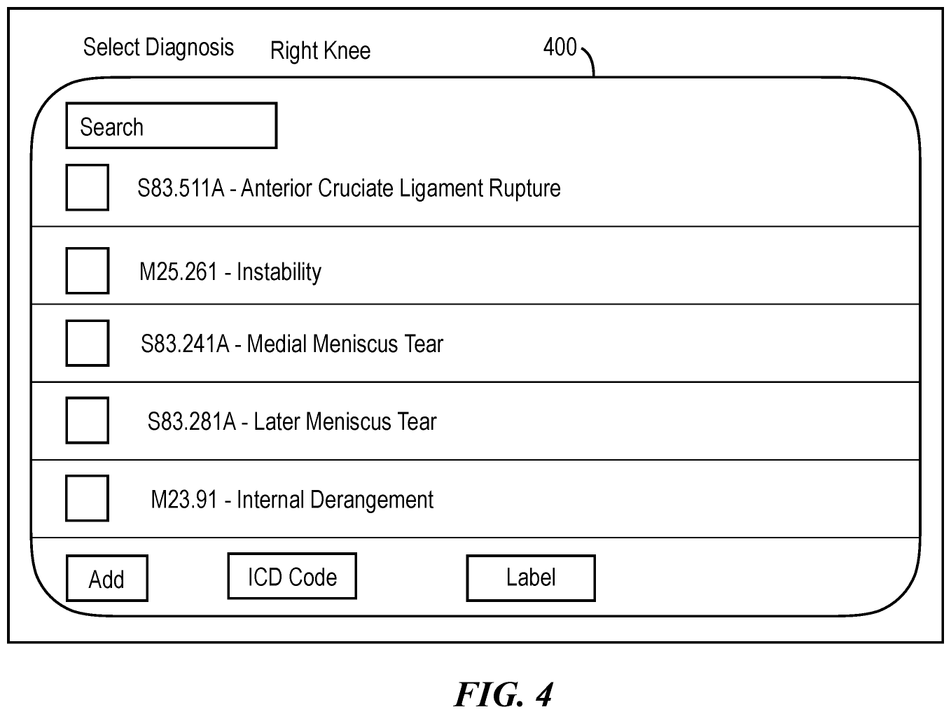

FIG. 4 illustrates a diagnosis selection interface 400 wherein the healthcare professional inputs a diagnosis corresponding to a patient. The healthcare professional selects from a listing of diagnoses such as anterior cruciate ligament rupture, instability, medical meniscus tear, lateral meniscus tear, internal derangement, etc. One skilled in the arts will readily understand that while the above listed diagnosis relate to a knee, the diagnosis may relate to any body part or point of injury or disfunction.

In some embodiments, the artificial intelligence engine anticipates and suggests diagnoses' using historical scheduling data for a particular context.

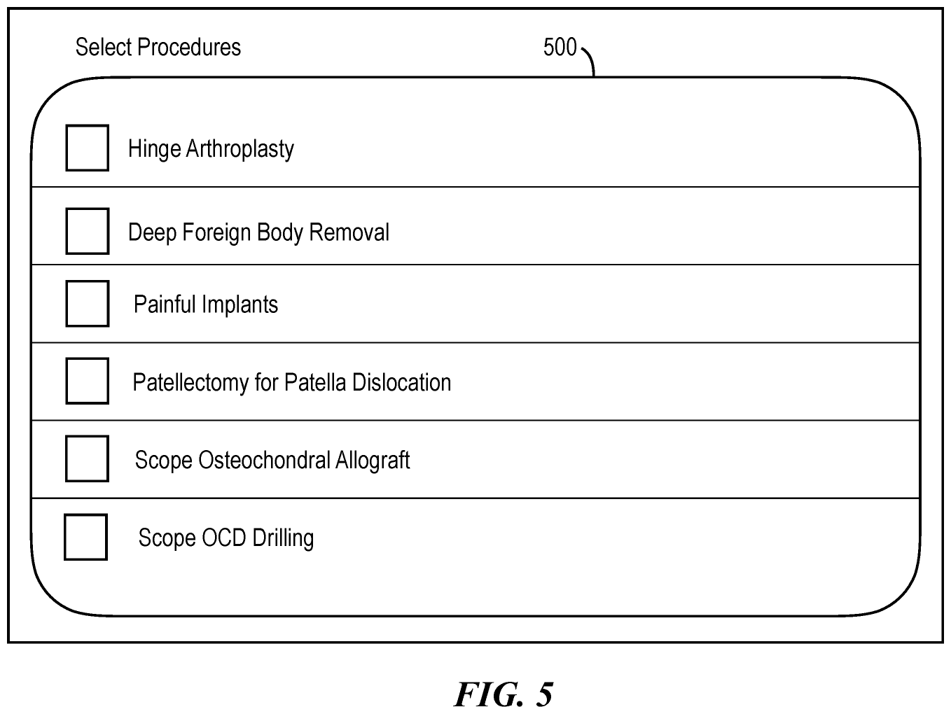

FIG. 5 illustrates a screenshot of the procedure selection interface 500 wherein the healthcare professional inputs patient information and procedure information corresponding to the patient. One or more procedures may be selected which automatically enters the current procedural terminology (CPT) number (or other billing code) corresponding to the procedure. For example, the healthcare professional may select procedures (e.g., hinge arthroplasty, deep foreign body removal, painful implants, patellectomy for patella dislocation, scope osteochondral allograft, scope OCD drilling, quad recon with graft, etc.). One skilled in the arts will readily understand that any procedure may be selected depending on the patient's needs.

In some embodiments, the system may anticipate and suggest what procedures the patient may qualify for based on insurance and/or clinical history published in existing notes, imaging studies, etc.

In some embodiments, the procedure selection interface may be automatically generated based on the diagnosis selection input by the healthcare professional or abstracted from the context of existing clinical records. Once selected, the healthcare professional can send the patient case to a scheduler.

In some embodiments, the healthcare professional may then manually or autonomously (via a scheduling module) schedule the procedure. In such, the healthcare professional may indicate whether they are using a date selector tool, the schedulers discretion when scheduling the procedure, or if the patient is undecided. If the patient is undecided, the patient is provided additional educational material and curated information to convert them to surgery. Further, the healthcare professional may input the priority level (e.g., high, medium, or low) wherein a high priority should be done as soon as possible, a medium priority can be done within a specific time period (e.g., within two weeks), and low priority indicates an elective surgery.

In some embodiments, the interface accounts for all logistical requirements for the surgery and can make some suggestions to the patient of available dates. Further, the interface may make requests for the scheduler to consider.

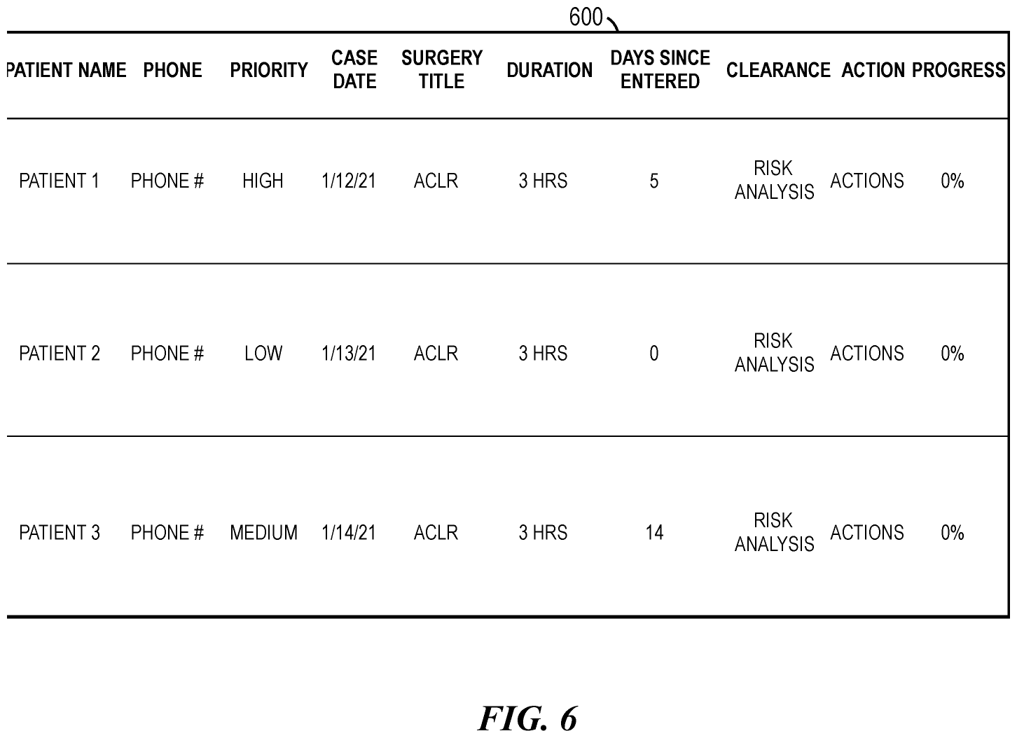

FIG. 6 illustrates a screenshot of the case listing interface 600 wherein the physician or other healthcare professional can view a listing of procedures which have been scheduled. For example, the case listing interface 600 may include patient names, contact information (e.g., phone number, address, email address, etc.), priority level, proposed case date, surgery title (i.e., procedure selected in FIG. 5), duration of the procedure, days since entered into the system, clearance status, actions, and progress. The clearance status may indicate any risks associated with the patient. Risks may be entered by the healthcare professional, abstracted from the medical record via the artificial intelligence engine, or directly solicited from the patient via an electronic questionnaire, and may include medical risk factors (e.g., medicine currently taking, active dental problems, breathing problems, kidney problems, liver problems, bleeding problems, diabetes, history of stroke/cerebrovascular accidents, obesity, history of infection, whether or not they are a smoker, chronic pain medication user, history of cancer, etc.) which may affect the procedure. Information related to a cardiac assessment may also be input.

In some embodiments, the system autonomously collects the clearances by engaging the patient, informing them they need to visit with recommended consultants, and then follow up with the patient to ensure the patient has been seen. The system may consolidate their recommendations through natural language processing to ensure the patient is appropriately prepared for planned procedures.

In some embodiments, the healthcare professional may also enter scheduling preferences, clearances, risk analysis information (as described hereinabove), relevant documents, status of the patient, or the ability to cancel the procedure. Further, insurance information may be input including primary and secondary insurance provider names and associated group numbers. The system may communicate information to and from the insurance company and indicate if authorization has been received from the patient's insurance provider.

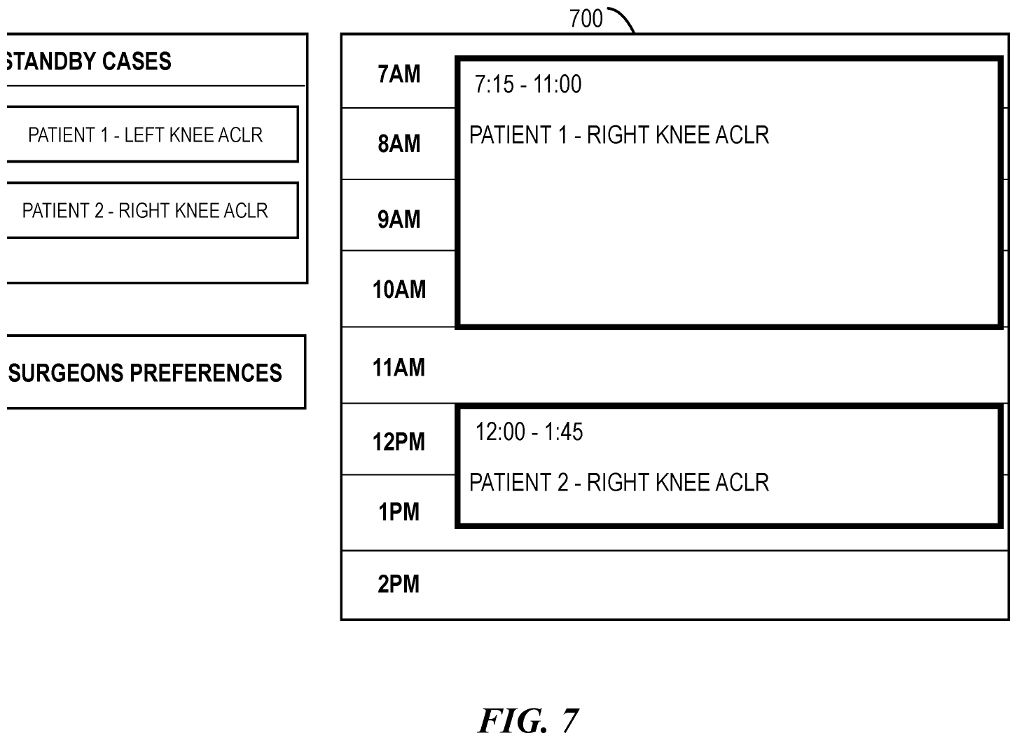

FIG. 7 illustrates a screenshot of the calendar interface 700 which illustrates a daily calendar showing scheduled procedures for a particular healthcare facility. In such, the healthcare professional can view available periods of time during which they may schedule additional procedures. This allows the healthcare professional to efficiently schedule each operating room (or other area of the healthcare facility) to help conserve operating costs. The calendar interface 700 may be used by a single healthcare professional or for a healthcare facility having a plurality of healthcare professionals each scheduling procedures. The user may have a listing of standby cases which can be dragged and dropped into the calendar interface 700. In some embodiments, the surgeons (or other healthcare professionals) preferences can be added to the calendar interface 700. The preferences may include schedule availability, procedural preferences, operating room preferences, etc. The preferences may also be used to automatically generate the optimum schedule accounting for surgeon-specific, patient-specific, case-specific, location-specific, or insurance-specific requirements. Each procedure provided on the calendar may be color-coded to indicate an aspect of the procedure such as the type of procedure or priority level.

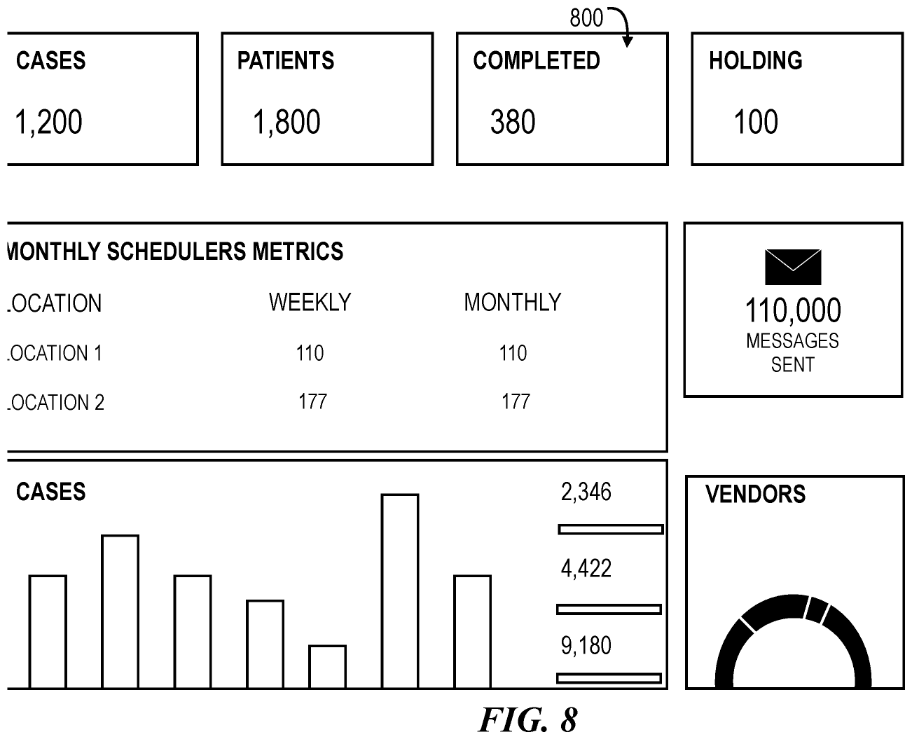

FIG. 8 illustrates a screenshot of the reports interface 800 to provide metrics for one or more healthcare facilities. The metrics may include total cases, total patients, completed procedures, cases which are holding, metrics for each healthcare facility (which may be split into monthly, weekly, or annual time periods), total communications sent, vendor information, and the like. In one example, the reports interface 800 may be used by administrative personnel to assess productivity of each healthcare facility.

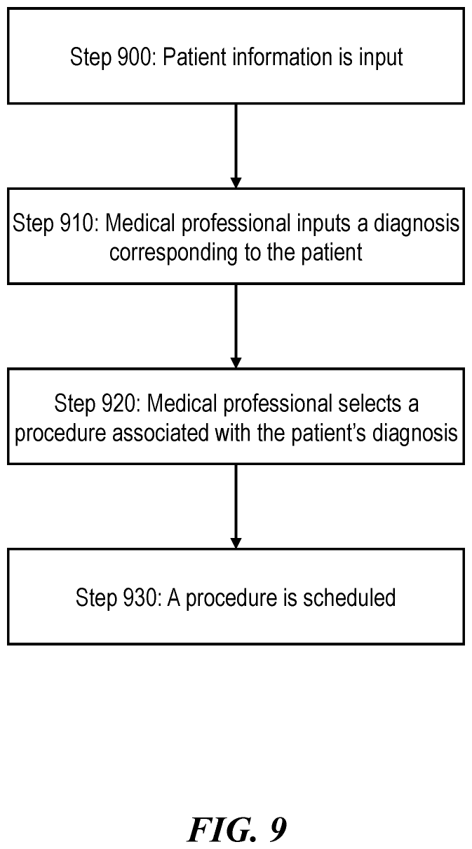

FIG. 9 illustrates a flowchart for the patient intake and scheduling process. Once the patient has a consultation with the medical professional the medical professional inputs the patient's information into the system in step 900. In step 910, the medical professional inputs a diagnosis which is corresponded to the patient. The diagnosis may be inferred by an artificial intelligence engine, such as by receiving the patient's symptoms and processing the symptoms to generate a diagnosis. Alternatively, the medical professional may select a diagnosis from a listing. In step 920, the medical professional inputs a procedure associated with the patient's diagnosis. The artificial intelligence engine may infer the procedure based on the patient's diagnosis. In step 930, the procedure is scheduled by the physician, a scheduler, or another medical professional.

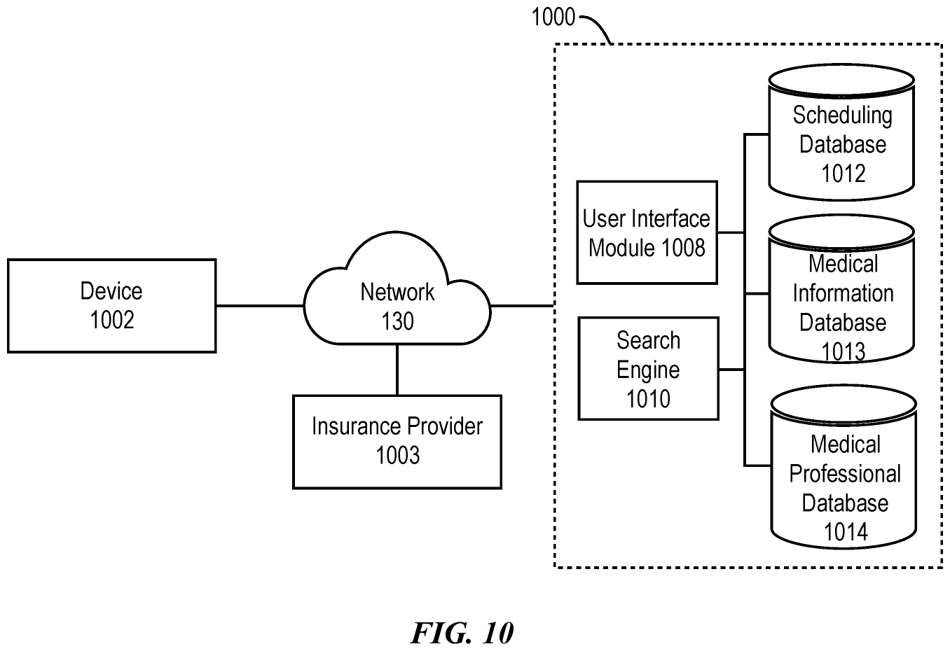

FIG. 10 illustrates a block diagram of the application system 1000 in operable communication with a device 1002 via a network 130. The device may include a computer, laptop computer, smartphone, tablet, or similar device which may be used to communicate with the patient, schedulers, insurance providers 1003, other medical professionals and users of the system. The application system 1000 includes a user interface module 1008 operable to display patient information, scheduling information, medical information, and other information made available by the device. The search engine 1010 permits the user to search information stored in the scheduling database 1012, the medical information database 1013, and the medical professional database 1014. The scheduling database 1012 stores scheduling preferences for each medical professional and medical facility. Further, the scheduling database 1012 stores scheduled procedures which have been scheduled via the system described hereinabove. The medical information database 1013 includes medical information which may be associated with each patient. The medical information may include diagnosis, symptoms, procedures, medical risk factors, etc. The medical professional database 1014 includes medical professional information including preferences, scheduling settings, specialties, etc.

In some embodiments, the insurance provider may automatically receive information related to the request for insurance as well as other relevant information related to the procedure. In such, the insurance approval process is made more efficient via the system described herein.

In some embodiments, the system creates a risk profile for each medical professional to potentially reduce the insurance costs billed to the medical professional. Based on the procedures being performed, the system also provides appropriate risk counseling information to patients, further reducing exposure to malpractice stemming from improper counseling of patients. For example, the medical professional may maintain a log of completed procedures and their outcomes. A sufficiently successful history of procedures may then result in the lowering of the medical professional's insurance premiums.

In some embodiments, the system may communicate with patients. In some embodiments, the system may solicit sound, video or text testimonials. The communication may be to provide advice to a patient currently undergoing therapy, or to ask for feedback related to the medical professional and/or healthcare facility. The feedback may be automatically formatted, branded, and edited before being transmitted to a social media profile associated with the patient and/or medical facility, or alternative marketing medium such as a website. The social media profile may be associated with the medical professional's personal information, professional profile, or contact information such that potential patients may interact with the medical professional's social media profile.

In some embodiments, the medical professional may select post-operative equipment which may be needed for the patient's recovery. This equipment may also be suggested by the AI engine. For example, a brace may be needed following a knee surgery. The system will schedule the ordering of the equipment to ensure the patient receives the equipment at an acceptable time.

In some embodiments, there may be specific requirement or tasks that a surgeon may have for a case, patient, or location that a facility may have for certain cases, surgeons, and patients, or a requirement when there is a certain type of patient. These tasks can be defined for any of the given criteria surrounding surgeons, patients, cases, insurances/payors, locations, or institutions that make it easy for oversight and control of the case management. For example, a surgical location may require all patients from a certain insurance to get a CT scan before their procedure. A task can be created for those cases that satisfy the defined criteria.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A medical procedure scheduling system, comprising:
a server comprising a server engine operable to communicate with a surgical scheduling management system comprising:
a point of care tool to permit a practitioner to construct at least one case, the case including case data;
a repository to receive the case data and retrieve one or more requirements for the surgical case;
a dashboard to disseminate the requirements and to transmit the requirements to at least one of a plurality of users via a communication system, and to transmit the requirements to a scheduler in operable communication with a scheduling system to generate a schedule for the case.

2. The system of claim 1, further comprising a risk analyzer configured to receive the case data, generate a risk profile, and correspond the risk profile to the case.

3. The system of claim 1, wherein the communication system comprises a notification generator to disseminate notifications to at least one of the plurality of users.

4. The system of claim 1, further comprising a pre-operation module to process pre-operation information and disseminate the pre-operation information to at least one of the plurality of users.

5. The system of claim 1, further comprising an operation module to process operation information and disseminate the operation information to at least one of the plurality of users.

6. The system of claim 1, further comprising a post-operation module to process post-operation information and disseminate the post-operation information to at least one of the plurality of users.

7. The system of claim 1, further comprising a recovery module to process recovery information and disseminate the recovery information to at least one of the plurality of users.

8. The system of claim 1, wherein the system utilizes a scheduling system to generate a schedule comprising scheduling data for at least one medical facility.

9. A medical procedure scheduling system, comprising:
at least one device in operable connection with a network;
an application server in operable communication with the network, the application server configured to host an application system displaying medical information and scheduling information, the application system having a user interface module for providing access to the application system to the at least one device, the user interface in operable communication with a scheduling database to receive scheduling information, the user interface comprising:
a case listing interface to display scheduled procedures scheduled by a healthcare professional; and
a calendar interface to permit the scheduling of one or more operating rooms at a healthcare facility.

10. The system of claim 9, wherein the medical information includes a patient diagnosis listing.

11. The system of claim 10, wherein the medical information includes a procedure listing.

12. The system of claim 11, further comprising an artificial intelligence engine to infer a procedure from a diagnosis.

13. The system of claim 12, wherein the artificial intelligence engine is in operable communication with the scheduling database to schedule a procedure.

14. The system of claim 13, further comprising an insurance provider in operable communication with the network to receive the medical information and indicate if insurance accepts or denies the procedure.

15. The system of claim 14, further comprising a risk analyzer configured to receive the case data, generate a risk profile, and correspond the risk profile to the case.

16. The system of claim 15, wherein the communication system comprises a notification generator to disseminate notifications to at least one of the plurality of users.

17. The system of claim 16, further comprising a pre-operation module to process pre-operation information and disseminate the pre-operation information to at least one of the plurality of users.

18. The system of claim 17, further comprising an operation module to process operation information and disseminate the operation information to at least one of the plurality of users.

19. The system of claim 18, further comprising a post-operation module to process post-operation information and disseminate the post-operation information to at least one of the plurality of users.

20. A medical procedure scheduling system, comprising:
at least one device in operable connection with a network;
an application server in operable communication with the network, the application server configured to host an application system displaying medical information and scheduling information, the application system having a user interface module for providing access to the application system to the at least one device, the user interface in operable communication with a scheduling database to receive scheduling information, the user interface comprising:
a case listing interface to display scheduled procedures scheduled by a healthcare professional;
a calendar interface to permit the scheduling of one or more operating rooms at a healthcare facility; and
an artificial intelligence engine to receive a patient diagnosis and suggest a procedure, and wherein the artificial intelligence engine schedules the procedure based on an operating room's availability.
